(19) United States
(12) Patent Application Publication
(10) Pub. No.: US 2020/0155751 A1
(43) Pub. Date: May 21, 2020

(54) ASPIRATION STENOSIS AND METHOD THEREFOR

(71) Applicant: Stanley Batiste, Granite Bay, CA (US)

(72) Inventor: Stanley Batiste, Granite Bay, CA (US)

(21) Appl. No.: 16/685,982

(22) Filed: Nov. 15, 2019

Related U.S. Application Data
(60) Provisional application No. 62/768,013, filed on Nov. 15, 2018.

(51) Int. Cl. A61M 1/36

(52) U.S. Cl. CPC A61M 1/3655; A61M 1/3663

(57) ABSTRACT

A graft with adjustable stenosis having a length of tube having tube wall with a central passage between an inlet end and an outlet end. A flexible barrier is located in the length of tube, that, in a default position, forms a narrowed section in the central passage and establishes a stenosis fluid chamber between the tube wall and the flexible barrier. An aspiration port system includes a port reservoir, containing fluid, and a needle entry seal, for needle access to the port reservoir to remove or add fluid. A channel is in fluid communication with the stenosis fluid chamber, the stenosis fluid chamber containing stenosis fluid chamber fluid. A separator is between the channel and the port reservoir. The flexible barrier is biased to a default narrow position. Responsive to fluid being removed from the port reservoir, the flexible barrier moves to increase the diameter of the narrowed section.

### 1. FIELD OF THE INVENTION

The invention relates generally to dialysis grafts and in particular to an adjustable graft using aspiration.

### 2. RELATED ART

There are currently more than 400,000 patients in the United States with end-stage renal disease (ESRD) and many times more than that throughout the world. ESRD accounts for approximately 6.4% of the overall Medicare budget at over $23 billion dollars in the US in 2006. Patients with end stage renal disease have lost their normal kidney function and as a result require dialysis to substitute the function of the kidney cleansing the blood. There are two types of dialysis; hemodialysis and peritoneal dialysis. For purposes of this overview we will primarily be focused on hemodialysis and later discuss briefly the topic of peritoneal dialysis.

Hemodialysis requires that large volume blood access and exchange be consistently available to sustain the life of the patient. Typically, a dialysis patient will require 3-4 hours of dialysis three days a week. The challenge with providing hemodialysis is maintaining access to large volumes of blood when a body constantly fights attempts to keep access available by healing closed such access. Currently there are three ways to provide hemodialysis; dialysis catheters, arterial venous fistulas (AVF's) and arterial venous grafts (AVGs). Although used worldwide, catheters are known not to be efficient for long term dialysis. Unfortunately, catheters have very short patency rates and high rates of infection. For these reasons, dialysis guidelines strongly oppose catheter use, other than short term, until fistula or graft placement is available.

AVG's and AVF's are synthetic and natural conduits respectively that are surgically placed to provide long term dialysis access. Both provide large diameter targets that can be easily accessed with large needles for blood exchange. These conduits are commonly placed in the arm with the furthest point attached to the patent's artery and then are directly attached to the vein for blood flow return. The high arterial blood pressure and flow is shunted directly to the vein providing dilatation of the vein or graft and large volume blood flow. Although these methods provide excellent means of access both have limitations with regard to sustaining long term patency. The patency rates are much greater than that of a catheter, however overall are relatively poor when considering the few years gained in a patient's life. It has been noted that there is only 50% shunt patency at one year and less than 25% at 2 years. Not only does this create a huge burden on the cost of healthcare but more importantly, once access is no longer available, a new access point must be created to sustain a patient's life.

A thorough description of the reason for dialysis fistula and graft failure is beyond the scope of this document. The fundamental problem is that the flow dynamics created by these artificial conduits are not normal to our bodies. The change is detected by the body and the normal physiologic defenses become involved and attempt to return the system to normal leading to graft or fistula failure. Failure of the graft generally means that the graft or fistula ceases to maintain flow. Once occluded, the grafts become full of blood which is static, and which subsequently becomes thrombus. Once failure occurs, the patient loses the ability to have hemodialysis until function is maintained.

From the discussion that follows, it will become apparent that the present invention addresses the deficiencies associated with the prior art while providing numerous additional advantages and benefits not contemplated or possible with prior art constructions.

### SUMMARY OF THE INVENTION

It has been shown that surgical improvements to failing dialysis grafts can improve long term patency and as a result, increase the lifespan of patients. One successful surgical procedure involves creating a narrowing within the mid aspect of a dialysis graft or fistula. This procedure is referred to as banding and requires that the graft is surgically exposed, and a suture is then applied around the graft and tightened narrowing the lumen creating a stenosis. This stenosis decreases pressure, flow and pulsation improving the hemodynamic properties of the fistula or graft. Although there are currently means of creating the stenosis this invention is unique in its construction and method of use.

The invention is a preformed stenosis which is either placed within or created from a standard dialysis graft or fistula. The stenosis is designed so that when in its neutral position it is the narrowest and cannot exceed a preset narrowing at any time. The stenosis will be made smaller than 50% of the standard graft or fistula diameter in its neutral position so that it can modify the hemodynamics. Once positioned within the graft the prescribed stenosis will improve flow characteristics. The design further considers the possibility of graft or fistula decreased function or occlusion with the need to adjust the stenosis to increase flow or open the stenosis up in order to clear the clot of thrombus. The invention therefore has a fixed stenosis the narrowing of which cannot be exceeded limiting operator error and a means of increasing the diameter of the stenosis to increase flow or have the capability to clear the dialysis graft of thrombus if occluded. The means to increase the stenosis from its neutral narrow position to a larger diameter opening is inherent to the invention design using negative pressure on the outside of the fixed stenosis drawing the walls outward stretching the inner opening. This negative pressure can be activated manually by such means as a syringe or mechanically both creating suction outside of the created stenosis. The method of the invention would involve placing the graft with the stenosis in its neutral narrow position and if flow adjustment was needed, or the graft needed to be cleared, the operator would open the stenosis by the described means of negative pressure. Once the graft was cleared, the operator can release the negative pressure allowing the stenosis to return to its neutral narrowed state.

To overcome the drawbacks of the prior art and provide additional benefits and features, a graft with adjustable stenosis is disclosed. In one embodiment, the graft includes a length of tube having tube wall with a central passage between two ends, an inlet and outlet. When in a default position, the flexible barrier inside the tube length forms a narrowed section in the central passage and a stenosis fluid chamber in between the tube wall and the flexible barrier.

The aspiration port system, in one embodiment, includes a port reservoir which is accessible through the needle entry seal that is configured to contain, add or remove port reservoir fluid from the chamber. This channel is in fluid communication with the stenosis fluid chamber which contains stenosis fluid chamber fluid.

In one configuration, the flexible barrier in the aspiration port system is biased to the default narrow position and therefore responsive to the removal of fluid from the port reservoir, moving to increase or decrease the narrowed section diameter. The flexible barrier may be constructed from rubber, latex, silicon or any combination thereof.

In this configuration, the separator is configured to prevent the overfilling of the port reservoir of the adjustable stenosis graft. The flexible barrier is biased such as if fluid leaks from the stenosis fluid chamber the section is maintained narrow. Removing fluid from the port reservoir pulls the separator into the port reservoir, which in turn pulls stenosis fluid chamber fluid into the port reservoir. The narrow section diameter increase is related to the amount of fluid removed from the port reservoir.

In another embodiment, the graft in the adjustable stenosis is configured to have a tube with an inner passage having a first diameter, and the tube having a wall with a first and second end. A second diameter of an inner lumen, which connects to the wall, is located inside the tube and is less than the first diameter. An inner lumen reservoir containing fluid is formed between the tube wall and inner lumen.

The aspiration port system comprises a first reservoir containing fluid accessible through an access port. The second reservoir is a channel connecting the inner lumen reservoir and the second reservoir. The aspiration port system also includes a separator dividing the first from the second reservoir.

In this configuration, removing the fluid from the first reservoir moves the separator which draws inner lumen reservoir fluid out of the inner lumen reservoir and into the aspiration port system. The removal of fluid from the inner lumen reservoir then changes the diameter of the inner lumen to a third diameter. This third diameter is between the first diameter and the second diameter. The fluid in the graft may be liquid or gas.

In this variation, the inner lumen, which is comprised of flexible material, a portion of which is biased to the second diameter, tapers from the first to the second diameter, and the access port is self-healing and further configured to be accessed by a needle.

The method for adjusting a diameter in a graft containing a stenosis, includes providing a tube that which has an inner passage having a first diameter. The tube with the wall an inner lumen is located inside the tube such that the inner lumen has a second diameter that is less than the first diameter and the inner lumen connecting to the wall. The inner lumen reservoir containing fluid is formed between the tube wall and inner lumen.

The aspiration port system may have a first reservoir containing fluid, accessible through an access port, a second reservoir, and a channel connecting the inner lumen reservoir and the second reservoir. A separator is further included dividing the first from the second reservoir.

The configuration of this system requires inserting a needle attached to a syringe into the access port, drawing the first reservoir fluid into the syringe through the needle, which lowers a pressure in the first reservoir relative to the second reservoir, which then moves the separator and draws fluid from the inner lumen. This method pulls the inner lumen from a default narrowed position to a less narrowed position.

This method further comprises injecting fluid through the access port into the first reservoir to increase pressure in the first reservoir, which in turn moves the separator, which then pushes fluid into the inner lumen reservoir causing the narrowing of the stenosis to have the second diameter.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The components in the figures are not necessarily to scale, emphasis instead being placed upon illustrating the principles of the invention. In the figures, like reference numerals designate corresponding parts throughout the different views.

FIG. 1 illustrates a dialysis machine 100 connected to a patient having a graft.

FIG. 2 illustrates a dialysis graft with stenosis and having an aspiration port system.

FIG. 3 illustrates the stenosis components with inflow lumen and a stenosis with narrowed inner lumen.

FIG. 4 illustrates a graft with an aspiration port system including a needle entry seal and reservoir, with a reservoir separator unit.

FIG. 5 illustrates the components in the neutral position with fluid within a syringe and the reservoir.

FIG. 6 illustrates components during aspiration with the syringe plunger creating negative pressure in the stenosis reservoir which pulls the separator and increases the volume in an adjacent reservoir.

FIG. 7 illustrates further aspiration achieved by further withdrawing the syringe plunger leading to lower reservoir pressures, which further increases the diameter of the lumen.

### DETAILED DESCRIPTION OF THE PREFERRED EMBODIMENTS

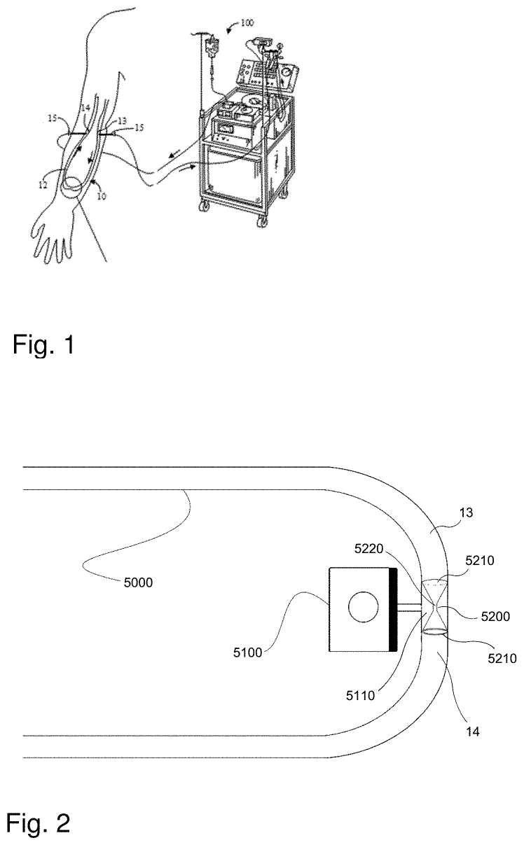

The invention is a means in which a preset narrowing can be placed or manufactured within a dialysis graft or fistula that can function in a neutral, non-stressed position to dampen the effects of high pressure, high flow and pulsation from upstream arterial flow creating low pressure flow at the graft or fistula outlet. This pressure regulation invention has a maximum narrowing aperture that cannot be further narrowed eliminating operator error yet, can be expanded allowing flow regulation and graft maintenance. FIGS. 1-5 are discussed jointly below.

As can be seen by reference to the drawings, and in particular to FIG. 1, the improved A-V graft construction that forms the basis of the present invention is designated generally by the reference number. Prior to embarking on a detailed description of the improved graft construction, the conventional graft construction is described. The graft can be used with standard equipment 100 in virtually all modem hemo-dialysis procedures.

As shown in FIG. 1, the prior art graft construction 11 includes an elongated length of hollow polymer tubing 12 having a uniform inside diameter extending from the inlet end 13 to the outlet end 14.

In addition, the conventional graft construction 11, as well as the improved graft construction 10, are commonly surgically placed within a patient's upper arm or forearm and connected via access needles 15 to a hemo-dialysis machine 100 that withdraws blood from the arterial end 13 and removes impurities from the blood prior to re-introducing the cleansed blood through the venous end 14. The arterial end 13 and the venous end 14 are on opposite sides of the stenosis.

As was mentioned previously, the hemo-dialysis procedure, requiring abnormally high blood flow rates through the conventional uniform internal diameter graft constructions 11, and the presence of the conventional graft construction 11, allows the elevated blood flow rates to continue unsub-sided during those periods when the access needles 15 are not connected to the hemo-dialysis machine 100.

As a direct consequence of these elevated blood flow rates, increased cardiac demands are imposed on the heart as blood is bypassed past the distal circulation. Further, the high flow rates result in venous irritation leading to stenosis and occlusion which typically occurs at the venous anastomosis.

As a consequence of the foregoing situation, and as shown in FIG. 2, the improved graft construction of the present invention includes a tubular stenosis structure 5200 having an inlet end 13, an outlet end 14, and a reduced diameter intermediate portion 5220 which forms the stenosis in a nature or artificial vessel 5000.

For description purposes the design can be described in two components as shown in FIG. 2, an aspiration port system 5100 for regulating the degree of narrowing in the stenosis structure 5200. The second component is the stenosis reservoir 5240 that creates the narrowing 16 which is located with the dialysis graft.

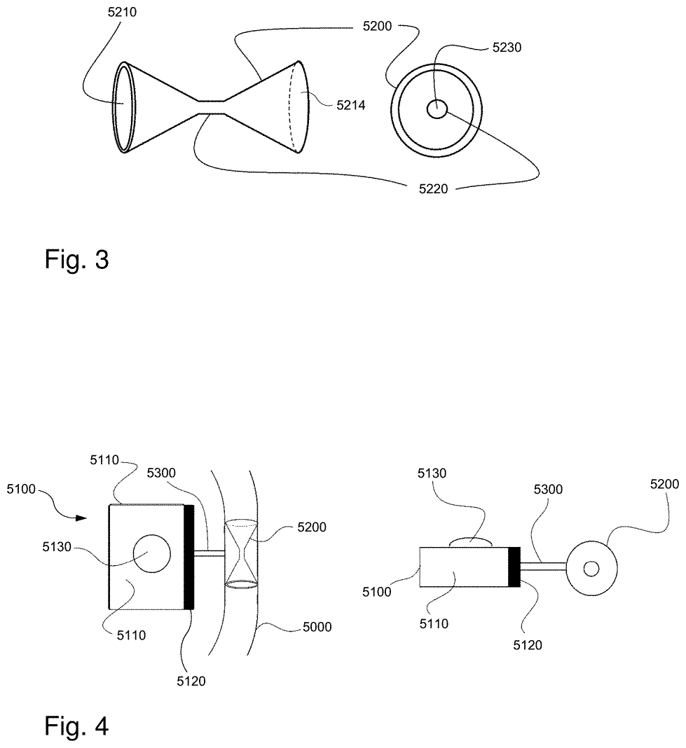

The stenosis structure 5200 is shown isolated in FIG. 3, where the end lumen 5210 is shown and has a similar second end lumen 5214 on the opposite end. Between the end lumens 5210, 5214, is the stenosis 5220 having a narrowed inner lumen 5230 which at its neutral position is less than 1/of the diameter of the end lumen 5210. The distance between the outer lumen and the inner lumen is gently tapered from the greater diameter opening toward the reduced diameter narrow section throughout its 360 degree arc or circumference of the tube, creating a smooth transition for fluid flow.

Blood flow from the patient enters the dialysis graft 5000 (shown in FIG. 1) then continues through the end lumen 5210 making a smooth transition to the narrowed inner lumen 5230 then a smooth transition back out through the second end lumen and through the dialysis graft. This transition and stenosis 5220 creates flow resistance which in turn decreases flow rate, pressure, and pulsation such that the fluid exiting the dialysis graft 5200 will have laminar flow with pressure near that of the normal human venous pressure.

In order to achieve regulation of the stenosis without the possibility of an event in which the stenosis becomes too narrow and occludes, the graft with aspiration port system 5100 is coupled with the stenosis as shown in FIG. 4. The aspiration port system 5100 has a main body 5150 and a port reservoir 5110 within the main body. The port reservoir 5110 can be accessed with a needle through the needle entry seal 5130. The needle entry seal 5130 reseals itself after access with a needle. Needle entry seal 5130 are known in the art and as such are not described in detail herein. The needle entry seal 5130 may be located at any location suitable for entry by a needle. The housing 5150 may be under the skin of a patient or external to the patient. Within the main body 5150 there is a piston unit referred to as a separator 5120 that is movable and forms a barrier between the port reservoir 5110 and the space leading to the graft including the connecting tube 5300.

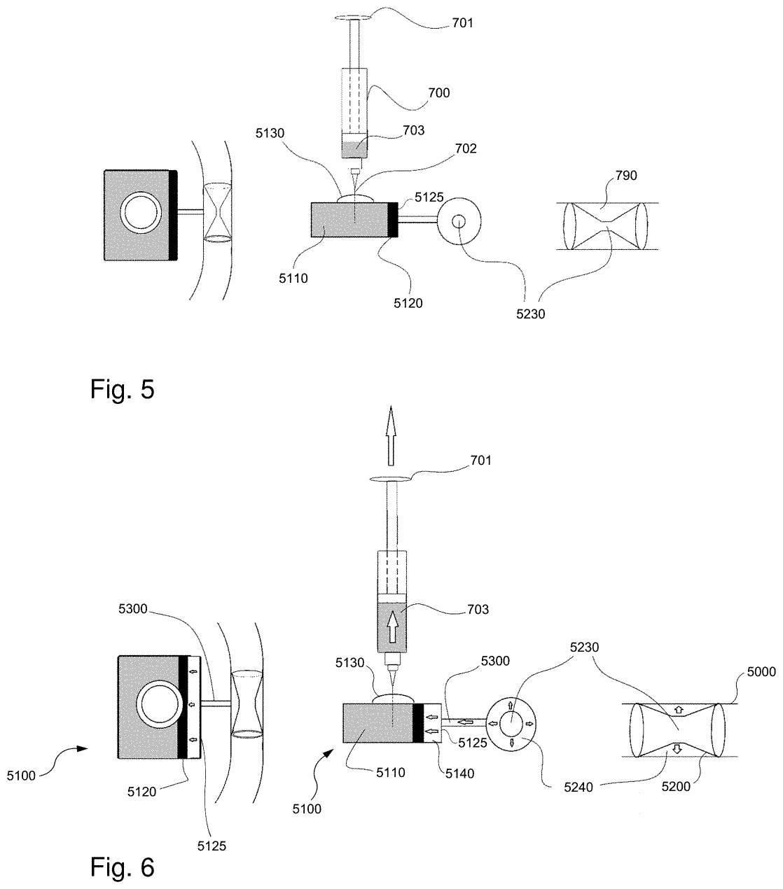

As shown in FIG. 5, a syringe 700 with needle tip 702 is used to access the port reservoir 5110. FIG. 5 demonstrates the system in its neutral operating position with the separator 5120 positioned against the wall of the aspiration port system 5100. Fluid is shown both in the syringe 703 and the port reservoir 5110 in grey. In normal or typical graft operation, the stenosis is at its maximum narrowed position, and if increased pressure is introduced by the operator using the syringe, the pressure will increase within the port reservoir 5110 but will not be passed on beyond the separator component protecting from creating too great a narrowing or occlusion. Thus, in the default position, such as without any reservoir pressure, the graft is in a narrowed position. Thus, if there is no pressure (positive or negative) in the fluid reservoir around the narrowing of the graft, the narrowing is shown in FIG. 5. This provides the benefit that if there is a leak or other anomaly over time which causes the reservoir 709 around the stenosis to not maintain pressure, the stenosis will stay narrowed thereby maintaining the pressure differential between each side of the graft, which in turn maintain blood pressure sufficient to supply blood to extremities.

If maintenance is required to clear clotting or blood accumulation or if there is need for additional flow through the graft, the stenosis 5230 can be widened or increased in diameter as shown in FIG. 6. Increasing the aperture (diameter) of the stenosis 5230 is accomplished by inserting the needle of the syringe into the access port 5130 and then engaging the syringe plunger 701 by pulling the plunger outward, which in turn lowers the pressure in the syringe and as a result pulls fluid in to the syringe 703, decreasing the fluid in the port reservoir 5110 of the aspiration port system 5100. This decreased fluid in the port reservoir 5110 creates decreased pressure in the reservoir causing the separator 5120 to move away from the wall 5125 of the aspiration port system 5100 creating decreased pressure in the connecting tube 5300. Although described as a tube 5300, the connecting path may be any shape or design. In this configuration, the separator 5120 is next to or in contact with the wall 5125 when the stenosis is at the narrowest diameter. This prevents the overfilling of the port reservoir 5110 which would reduce the stenosis to an overly narrow a diameter.

As can be appreciated, the amount of fluid in the connecting tube 5300 and the stenosis fluid chamber 5240 determines or influences the diameter of the stenosis. If additional fluid is added to the connecting tube 5300 and the stenosis fluid chamber 5240, then the stenosis will become narrower. Conversely, if less fluid is added to the connecting tube 5300 and the stenosis fluid chamber 5240, then the stenosis will become less narrow. In one embodiment, there is an access port that can be used to add or remove fluid to the connecting tube 5300 and the stenosis fluid chamber 5240 at set up or just prior to placement of the graft with stenosis in the patient. This can occur to fine tune the amount of narrowing based on the patient's vessel diameter and the surgeon's professional judgement.

It also disclosed that the separator may be biased in some manner to push the fluid into the area 5240 thereby creating a default position for the stenosis to be at maximum narrowing and prevent the separator, and the fluid linked stenosis diameter from moving without use of the needle/syringe. This bias may be a spring or any other bias device.

When the term pressure is used herein, it is in relation to and relative to another pressure. Thus, assuming an equilibrium between the fluid in port reservoir 5110 and stenosis space area 5240, when the syringe plunger is drawn backwards, fluid is drawn into the syringe, thereby lowering the pressure in port reservoir 5110 relative to the pressure of the fluid in area 5240. This pressure differential causes the separator 5120 to move to the left as shown in progression of FIGS. 5, 6, and 7. There is also blood pressure in the inner area of the graft/stenosis area that is pushing outward on the wall 5220 of the stenosis causing the narrowing to expand as the pressure in areas 5240 becomes less due to movement of the separator 5120. It should also be noted that in general, fluid, unlike a gas, does not compress.

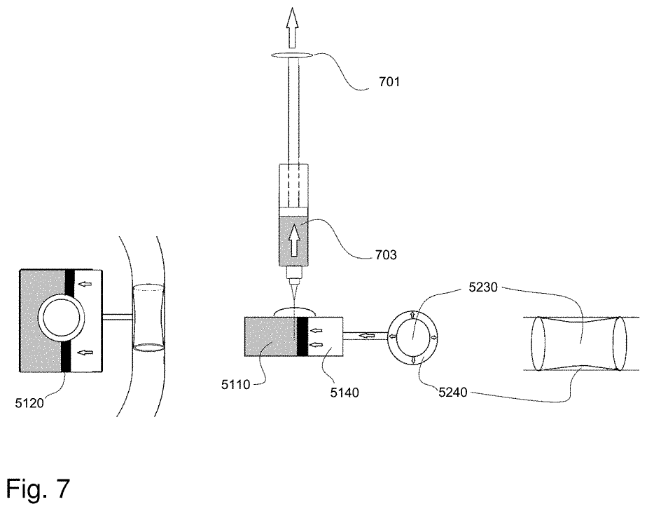

The decreased pressure in the connecting tube 5300 then decreases the pressure in the stenosis fluid chamber 5240 between the dialysis graft 5000 and the stenosis unit 5200. As fluid is aspirated from the stenosis fluid chamber 5240 between the dialysis graft 5000 (the wall of the graft) and the stenosis unit 5200, the stenosis unit stretches out due to the low pressure, and the inner lumen 5230 enlarges. The walls of the stenosis unit are biased to be in the narrow diameter configuration shown in FIG. 5. The reduced pressure overcomes the bias of the walls of the stenosis unit thus causing the stenosis to open in relation to the reduction in pressure in the stenosis fluid chamber 5240. The removal of the fluid in the stenosis fluid chamber 5240 stretches the wall of the stenosis unit outward to increase the diameter of the stenosis. The inner lumen is stretchable and flexible as it comprises a material such as rubber, latex, silicon or any other biocompatible elastic material. If the pressure were removed from the fluid in the stenosis fluid chamber 5240, the stenosis would return to the narrow position. FIG. 7 illustrates further syringe aspiration with further increase in inner luminal diameter 5230 as compared to FIG. 6. Throughout all the figures, identical elements are identified with identical reference numbers.

The fluid in the syringe and the port reservoir 5110 may be any type fluid or any type gas. Likewise, the fluid in the connecting tube 5300 and the stenosis fluid chamber 5240 may be any type fluid or any type gas.

A summary of the figures is as follows. FIG. 1 shows a dialysis machine 100 with inflow and outflow lines 15 and graft with proximal flow limb 12 and distal flow limb 10 having a mid-graft stenosis 5200. FIG. 2 shows the stenosis 5200 within the dialysis graft 5000 having an aspiration port system 5100. FIG. 3 is a drawing of the stenosis component 5200 with inflow lumen 5210 and stenosis 5220 with fixed narrowed inner lumen 5230. FIG. 4 shows the connected aspiration port system 5100 and the stenosis 5200 with needle entry seal 5130 within the port reservoir 5110, with reservoir separator unit 5120. Also shown is the commination tube 5300 leading to the space between the dialysis graft 5000 and the stenosis 5200. FIG. 5 illustrates the components in the neutral position with fluid within the syringe 703 and the port reservoir 5110 and the narrowed position of the stenosis 5230.

FIG. 6 shows aspiration by engaging the syringe plunger 701 creating negative pressure drawing in fluid 703 from port reservoir 5110 decreasing the volume pulling the separator 5120 and increasing volume in adjacent reservoir 5140. Fluid flows out of the space 5240 creating low pressure stretching the stenosis and increasing the size of the lumen 5230. FIG. 7 shows further aspiration by further withdrawing syringe plunger 701 leading to lower pressure in the port reservoir 5110 and 5140 increasing the diameter of the lumen 5230.

What is claimed is:

1. A graft with adjustable stenosis comprising:
a length of tube having tube wall with a central passage between an inlet end and an outlet end;
a flexible barrier, in the interior of the length of tube, that, in a default position, forms a narrowed section in the central passage and a stenosis fluid chamber between the tube wall and the flexible barrier;
an aspiration port system comprising:
a port reservoir, accessible through the needle entry seal, that is configured to contain port reservoir fluid;
a needle entry seal configured for needle access to the port reservoir to remove fluid from or add fluid to the port reservoir;
a channel in fluid communication with the stenosis fluid chamber, the stenosis fluid chamber containing stenosis fluid chamber fluid;
a separator between the channel and the port reservoir;
wherein the flexible barrier is biased to the default narrow position and, responsive to fluid being removed from the port reservoir, the flexible barrier moves to increase the diameter of the narrowed section.

2. The graft of claim 1 wherein the flexible barrier is formed from rubber, latex, silicon or a combination thereof.

3. The graft of claim 1 wherein the separator prevents overfilling of the port reservoir.

4. The graft of claim 1 wherein the flexible barrier is biased to for the narrow section such if fluid leaks from the stenosis fluid chamber the narrow section is maintained.

5. The graft of claim 1 wherein removing fluid from the port reservoir pulls the separator into the port reservoir, which in turn pulls stenosis fluid chamber fluid into the port reservoir.

6. The graft of claim 1 wherein the amount of diameter increase in the narrow section is related to the amount of fluid removed from the port reservoir.

7. An adjustable stenosis configured as part of a graft comprising:
a tube having an inner passage having a first diameter, the tube having tube wall, a first end and a second end;
an inner lumen located inside the tube, such that inner lumen has a second diameter that is less than the first diameter and the inner lumen connect to the wall;
an inner lumen reservoir, containing inner lumen reservoir fluid, the inner lumen reservoir formed between the tube wall and inner lumen;
an aspiration port system comprising;
a first reservoir, containing first reservoir fluid, accessible through an access port;
a second reservoir;
a channel connecting the inner lumen reservoir and the second reservoir;
a separator dividing the first reservoir from the second reservoir;
wherein removing first reservoir fluid from the first reservoir moves the separator which draws inner lumen reservoir fluid out of the inner lumen reservoir and into the aspiration port system, the removal of fluid from the inner lumen reservoir changing the diameter of the inner lumen to a third diameter, the third diameter between the first diameter and the second diameter.

8. The graft of claim 7 wherein the fluid is a liquid or a gas.

9. The graft of claim 7 wherein the inner lumen tapers from the first diameter to the second diameter.

10. The graft of claim 7 wherein the inner lumen comprises a flexible material, a portion of which is biased to the second diameter.

11. The graft of claim 7 wherein the access port is self-sealing and configured to accessed by a needle.

12. A method for adjusting a stenosis diameter in a graft containing a stenosis, the method comprising:
providing a tube having an inner passage having a first diameter, the tube having tube wall, an inner lumen located inside the tube, such that inner lumen has a second diameter that is less than the first diameter and the inner lumen connect to the wall, an inner lumen reservoir, containing inner lumen fluid, the inner lumen reservoir formed between the tube wall and inner lumen, and an aspiration port system comprising;
a first reservoir, containing first reservoir fluid, accessible through an access port, a second reservoir, a channel connecting the inner lumen reservoir and the second reservoir, and a separator dividing the first reservoir from the second reservoir;
inserting a needle attached to a syringe into the access port;
drawing first reservoir fluid into the syringe through the needle, the drawing of fluid from the first reservoir lowering a pressure in the first reservoir relative to the second reservoir, which moves the separator and draws inner lumen fluid from the inner lumen, which pulls the inner lumen from a default narrowed position to a less narrowed position.

13. The method of claim 12 further comprising injecting fluid through the access port into the first reservoir to increase pressure in the first reservoir, which in turn moves the separator, which pushes fluid into the inner lumen reservoir, which narrows the stenosis to the second diameter.
